(19) United States
(12) Patent Application Publication
(10) Pub. No.: US 2016/0270741 A1
(43) Pub. Date: Sep. 22, 2016

(54) MEDICAL MONITOR

(71) Applicant: Derak Joseph RANKE, East Dublin, GA (US)

(72) Inventor: Derak Joseph RANKE, East Dublin, GA (US)

(21) Appl. No.: 15/171,534

(22) Filed: Jun. 2, 2016

Related U.S. Application Data
(63) Continuation-in-part of application No. 14/139,329, filed on Dec. 23, 2013.

(51) Int. Cl. A61B 5/00; A61F 13/00; A61B 5/02

(52) U.S. Cl. CPC A61B 5/7445; A61B 5/746; A61B 5/02042; A61F 13/00055; A61B 2562/164; A61B 2562/166

(57) ABSTRACT

A medical monitor is provided. The medical monitor may include a transparent display sheet providing spaced apart first and second conductive elements along a distal side of the display sheet. Electric ink marks may electrically connect to either the first or the second conductive element so as to form an open circuit having spaced apart first and second electric ink markings along said distal side. The display sheet may be sandwiched between two bandage sheets prior to placing the proximal side near or around a wound site the open circuit was tailored for. Wherein blood from the wound site would migrate through the two bandage sheets onto the distal side, bridging the open circuit of adjacent first and second ink markings, so that if the open circuit where electrically connected in series with a power supply and an alarm, the alarm would be powered.

### CROSS-REFERENCE TO RELATED APPLICATION

This application is a continuation-in-part of application U.S. Ser. No. 14/139,329, filed on Dec. 23, 2013. The contents of which are herein incorporated by reference.

### BACKGROUND OF THE INVENTION

The present invention relates to medical monitoring systems and, more particularly, to alerting caregivers when a patient is bleeding.

Many devices have been proposed to detect when patients may be bleeding. Currently, such devices are expensive considering the dictates of the medical field that demand disposal of blood contaminated items after one use. Moreover, current devices involve many and sometimes bulky parts the preclude adapting the device effectively to problematic and otherwise hard to reach bleed sites.

As can be seen, there is a need for a medical monitoring system that can alert caregivers when a patient is bleeding and that is also adaptable for problematic and otherwise hard to reach bleed sites. Moreover, it is desirable and advantageous that the medical monitoring system be inexpensive given the mandated disposal and repurchase of the medical monitoring system after one use.

### SUMMARY OF THE INVENTION

In one aspect of the present invention, a medical monitor comprises: an alarm; a power supply; an open circuit comprising two spaced electrically conductive elements electrically connected in series with the alarm and the power supply; and a first sheet comprising a distal side and a proximal side, wherein the distal side is connected to the open circuit.

In another aspect of the present invention, a method of detecting blood comprises: providing a medical monitor comprising: an alarm; a power supply; an open circuit comprising two spaced electrically conductive elements electrically connected in series with the alarm and the power supply; and two sheets comprising a distal side and a proximal side, wherein the distal sides sandwich the open circuit; and mounting the two sheets over a bleed site.

In yet another aspect of the present invention, a blood sensor includes a display sheet having a proximal side and an opposing distal side; a first and second conductive element disposed along the distal side of the display sheet; a plurality of first electric ink markings extending from the first conductive element; and a plurality of second electric ink markings extending from the second conductive element, wherein none of the plurality of first electric ink markings touch any of the plurality of second electric ink markings, wherein the display sheet is transparent and made from flexible material adapted to conform to a body part of a patient, and wherein each first electric ink marking is spaced apart from each adjacent second electric ink marking, and further including a first bandage sheet interfacing the distal side of the display sheet; and a second bandage sheet interfacing the proximal side of the display sheet, wherein the first and second bandage sheets sandwich the display sheet, wherein the plurality of first and second electric ink markings and the first and second conductive elements provide an open circuit, and wherein the open circuit is electrically connected in series with a power supply and a power alarm.

In yet another aspect of the present invention, a method of detecting blood includes providing a transparent display sheet having a proximal side and an opposing distal side, wherein the transparent display sheet is made from a flexible material adapted to conform to a body part of a patient, and wherein a first and second conductive element is disposed along said distal side; marking said distal side with electric ink so as to configure a desired open circuit, wherein a first portion of the electric ink electrically connects to the first conductive element and a second portion of the electric ink electrically connects to the second conductive element; electrically connecting the open circuit in series with a power supply and a power alarm; and placing said proximal side near a wound site. And in certain embodiments, further sandwiching the display sheet between a first and second bandage sheet prior to placing said proximal side near the wound site, and further determining the desired open circuit configuration based in part on the wound site.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a plan view of an exemplary embodiment of the alarm circuitry of the present invention;

FIG. 3 is a plan view of an exemplary embodiment of the open circuit of the present invention; and

FIG. 4 is an exploded view of an alternative embodiment of the open circuit of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a medical monitor. The medical monitor may include a plurality of sheets and a power source electrically connected in series with an alarm and an open circuit. The plurality of sheets may include a distal side and a proximal side. The open circuit may be sandwiched between distal sides of adjacent sheets. A user may mount either proximal side to a bleed site on a patient where bleeding can occur, for example, entry points for intravenous applications. The plurality of sheets may allow blood to flow from the bleed site through the proximal side and through the distal side so that the blood may close the open circuit, so that the alarm is powered.

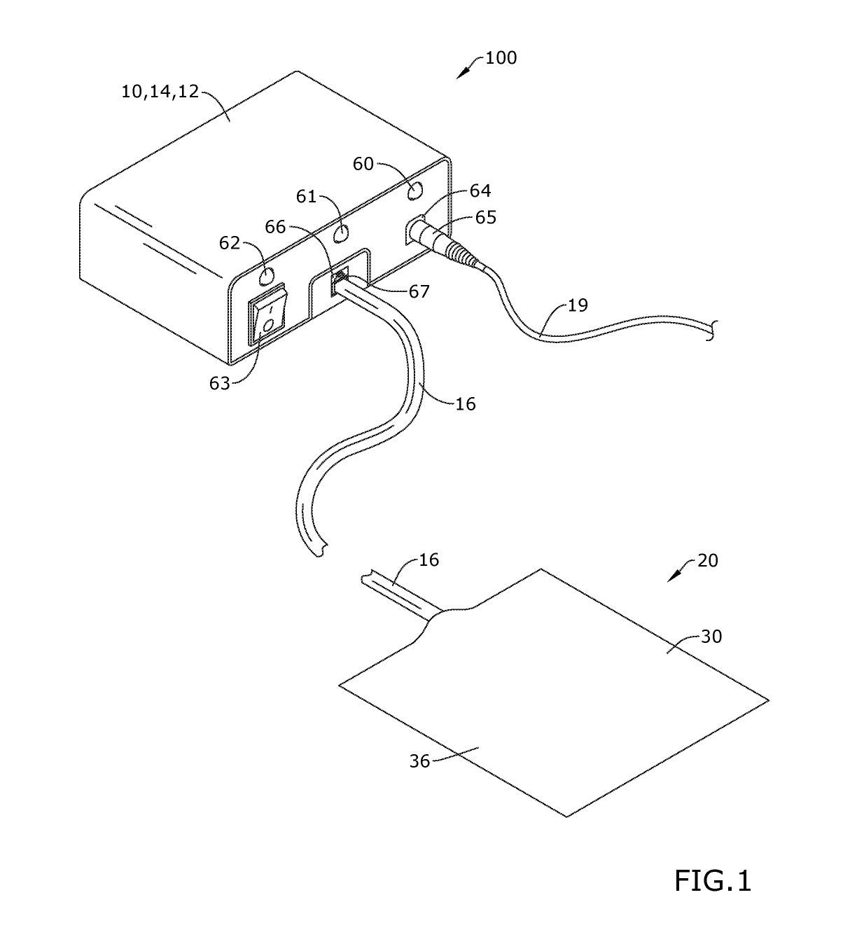

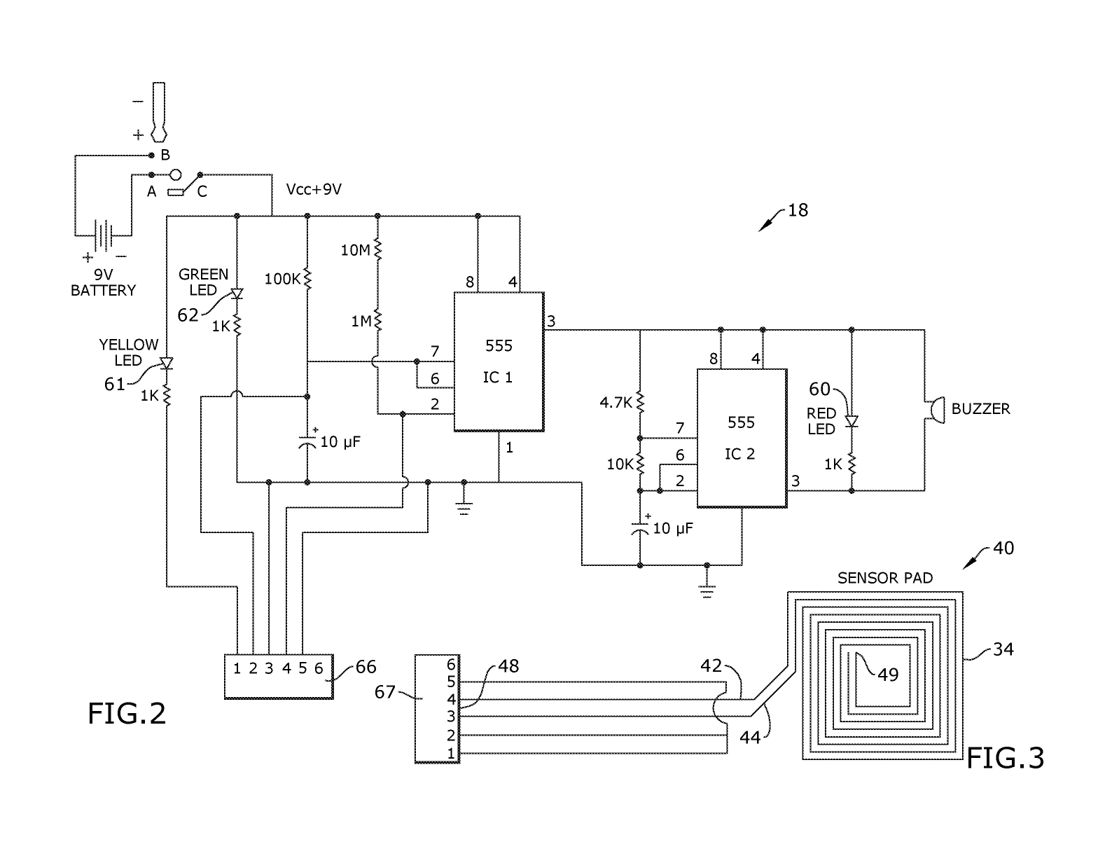

Referring to FIGS. 1 through 4, the present invention may include a medical monitor 100. The medical monitor 100 may include a power alarm 10 and a blood sensor 20.

The blood sensor 20 may include a plurality of sheets 30 sandwiching an open circuit 40. The plurality of sheets 30 may include porous, non-conductive material. The plurality of sheets 30 may include a distal side 34 and a proximal side 36. The proximal side 36 may be adapted for mounting directly on a patient. The distal side 34 may be adapted for encasing the open circuit 40.

The plurality of sheets 30 material may include gauze, bandage, fabric with a loose open weave, netting type structure and the like that is sufficiently pliable to adapt the blood sensor 20 to problematic and hard to reach bleed sites. The plurality of sheets 30 thickness and material may be sufficiently permeable so as to allow free flow of blood from and through the proximal side 36 to and through the distal side 34. The plurality of sheets 30 thickness and material may be sufficiently impermeable so as to prevent the free flow of non-blood fluid from and through the proximal side 36 to and through the distal side 34. The plurality of sheets 30 may have various shapes, such as, square, rectangular, circular, oval, trapezoidal, triangular, u-shape, etc.

In certain embodiments, the plurality of sheets 30 may be a collection of separate sheets 30 stacked together, wherein the distal side 34 of one sheet 30 removably attached to the proximal side 36 of an adjacent sheet 30. The removable attachment between adjacent sheets 30 may be the result of friction forces, adhesive substances, combinations thereof, and the like

The open circuit 40 may be held and supported by at least one distal side 34. The open circuit 40 may include a first element 42 and a second element 44. The first element 42 and the second element 44 may be electrical wire, electrical conductors and the like, that may be sufficiently malleable to adapt the blood sensor 20 to problematic and hard to reach bleed sites. The first element 42 and the second element 44 may run parallel and be spaced apart at a predetermined distance. The open circuit 40 may conform to the shape of the plurality of sheets 30 so that a portion of the open circuit 40 may be present over substantially all of the surface area of the distal side 34. The open circuit 40 may terminate at a port end 48 and at a sensor end 49. The port end 48 may include a plug 67.

In certain embodiments, the sensor end 49 may terminate near an opening disposed within the plurality of sheets 30. The opening may be mounted around the bleed site.

The power alarm 10 may include an alarm 12, a voltage source 14, an alarm circuitry 18, a cord 16, a jack 66 and a power source 19 for powering the power alarm 10. The alarm 12 may be of various types such as, but not limited to, noise-making, light-activating, vibration-emitting types and the like, sufficient to alert all necessary attendants.

In certain embodiments, the power alarm 10 may include an alarm light 60, a notification light 61, a power light 62, a power switch 63 and a power input port 64. The power source 19 may include a power jack 65 connectable to the power input port 64.

The cord 16 may electrically connect the blood sensor 20 to the power alarm 10. In certain embodiments, the cord 16 may terminate in the plug 67 connectable to the jack 66. The voltage source 14 may apply a potential difference between the first element 42 and the second element 44.

The alarm circuitry 18 may be configured so that when the first element 42 and the second element 44 circuit 4 the alarm 12 receives power. The alarm circuitry 18 may be configured for dialysis settings. In an alternative embodiment, the power source 19 may be the voltage source 14 electrically connected in series with the alarm 12 and the open circuit 40 so that blood may close the circuit so as to power the alarm 12.

A method of using the present invention may include the following. The medical monitor 100 disclosed above may be provided. A user may mount either proximal side 36 of the plurality of sheets 30 to a bleed site on a patient where bleeding can occur, for example, entry points for intravenous applications. The plurality of sheets 30 may allow blood to flow from the bleed site through the proximal side 36 and to and through the distal side 34 so that the blood may contact the open circuit 40. If sufficient bleeding occurs, the blood may bridge the predetermined distance between the first element 42 and the second element 44 so as to close the open circuit 40.

In certain embodiments, the user may add additional layer of the sheets 30 by mounting one sheet's 30 proximal side 36 to an adjacent sheet's 30 distal side 34. The additional layers may be used as an intermediate barrier between the bleed site and the open circuit 40 to adjust the sufficient of blood needed to close the open circuit 40.

In certain embodiments, the plurality of sheets 30 may be tailored in shape and size to accommodate various sizes and locations of bleed sites. In certain embodiments, the medical monitor 100 may be placed between the part of the patient's body part containing the bleed site and where that body part rests. In an alternate embodiment, the medical monitor 100 may be placed on the floor or bed where the patient is at rest.

From the description above, a number of advantages of some of the embodiments become evident. For example, the plurality of sheets 30 can be made from cost effective material, whereby a few rolls of it will permit the manufacturer to cost effectively mass produce the medical monitor 100. This would be a boon in the medical field where blood-contaminated devices are required to be thrown away after one use. Likewise, the medical monitor 100 is pliable and of adaptable construction, allowing a user to apply the present invention to problematic and hard to reach bleed sites. Moreover, either side of the blood sensor 20 may detect blood and may be mounted to the patient.

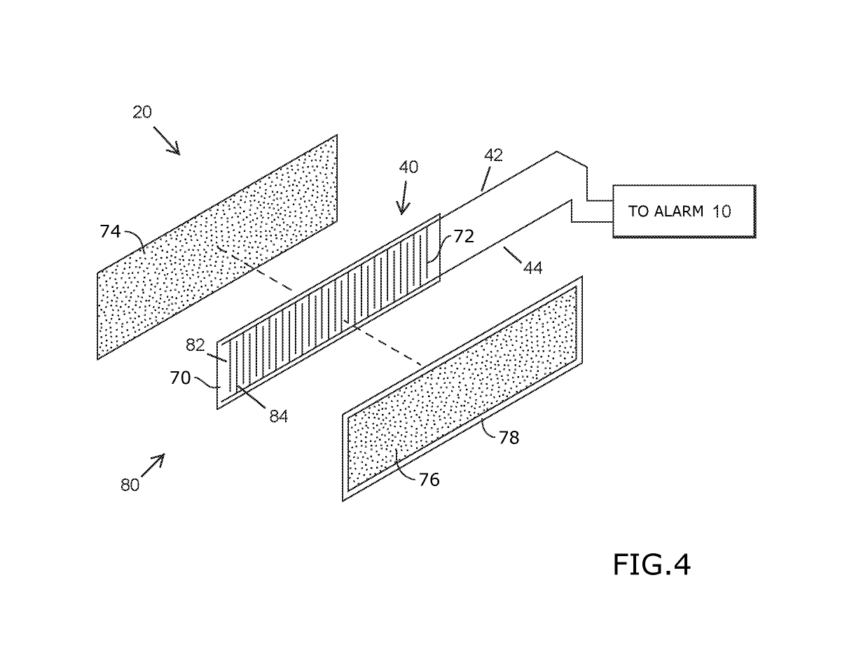

In another embodiment, the blood sensor 20 may be electrically connected to the power alarm 10 by the first element 42 and the second element 44, wherein the open circuit 40 is, in at least part, defined by electronic ink 72 provided on a display sheet 70, as illustrated in FIG. 4. The electronic ink 72 may be a medium that conducts an electric current, wherein medium may be in liquid form, making it “writable” on the display sheet 70, and wherein the medium occupies a solid form around room temperature, so when written and dried, may form a fixed electric network on the display sheet 70. Thereby, the electric network (here, the open circuit 40) may be formed and defined, if needed when confronted with a unique wound site, on demand into a desired formation or configuration.

In one instance, the electric network/open circuit 40 may be a plurality of first ink connectors/markings 82 and second ink connectors/markings 84, provided by the electric ink 72 electrically extending from either the first element 42 and the second element 44, respectively. Whereby, the first element 42 and the second element 44 may, in effect, be electrically extended over the display sheet 70 in the desired configuration/formation defined by the first ink connectors/markings 82 and second ink connectors/markings 84. The first and second elements 42, 44 may be electrically interconnected to the alarm box/power alarm 10 via the port end 48, plug 67, cord 16 or other sufficient electrical connections.

The requirement that the first and second elements 42 and 44 may only need extend in one direction, as illustrated in FIG. 4, and not bend or turn along the support sheet (“sensor pad”), as illustrated in FIG. 3. The decreased allowance on the first and second elements 42, 44 in terms of their overall length and tortuous nature allows for ease of mass production as well as flexibility of display sheet 70, which is not subject to demands and rigidity the framework of the tortuous first and second elements 42, 44. Also, electric ink 72 is rigidly connected to display sheet 70/elements 42, 44 and so less likely to become dislodged or move out of place and thus unwantedly closing the open circuit.

In the present invention, each first ink connectors 82 may maintain a spaced apart (in some embodiments, a parallel) relationship with each adjacent second ink connector 84. Such open circuit grids enable blood that is present on the display sheet 70 side providing the electric ink 72 to bridge adjacent first and second ink connectors 82, 84, thereby closing the open circuit 40 and empowering the power alarm 10, as discussed above. Advantageously, each ink connector 82 and 84 may be configured/formed in situ so as to conform with or accommodate a present wound site that the blood sensor 20 may be placed around or adjacent to. Thus the present invention lends itself to being adaptable to the patients' needs, as they arise and are unexpectedly presented to the healthcare provider. Furthermore, this adaptability is a boon when the blood sensor 20 needs to be altered to accommodate working with certain medical equipment, like a dialysis for example.

The use of electric ink 72 enables another advantage over the prior art, the display sheet 72 may be transparent, for example the display sheet 70 may be a transparent film, a plasticized membrane or sheet or the like, that facilitates the reception and retention of electric ink in accordance with the present invention as described herein. The transparency enables a healthcare provider to apply the blood sensor 20 to, near, adjacent to and around a wound site, possibly an inconveniently located would site, while seeing through the display sheet 70. As opposed to placing a prior art blood sensor wherein the substrate or material supporting the open circuit obscures the best or proper placement of the prior art blood sensor near the wound site. Moreover, the transparent sheets tend not to warp or wrinkle when the electric ink dries, yet maintain flexibility during use.

In certain embodiments, the display sheet 70 may be sandwiched by an opposing first and second bandage sheet, 74 and 76 respectively, as illustrated in FIG. 4. One of the bandage sheets may provide a peripheral adhesive 78 for sealing together a periphery of the first bandage sheet 74 to a periphery of the second bandage sheet 76, securing the sandwiched the display sheet 70 therein. The bandage sheets 74, 76 may be gauze or another translucent material that would let light through so as to further facilitate the placement of the blood sensor 20 at or near a wound site. The bandage sheets 74, 76 may also be permeable so that blood from the wound site will run there through so as to interface with the open circuit 40.

In certain embodiments, the blood sensor with the electric ink 72 on the transparent display sheet 70 (ink blood sensor 80 hereafter) may be used with the electric ink portion/side of the display sheet 70 facing away from the wound site. Thus the ink blood sensor 80 may have electric ink 72 on only one of the opposing sides of the display sheet 70. As a result, the transparent display sheet 70 may be an impermeable barrier between the patient's skin and the electric ink open circuit 40. This way, the display sheet 70 acts as a barrier so that body moisture will not inappropriately bridge the open circuit, producing a false alarm through the power alarm 10.

In certain embodiments, the first bandage sheet 74 interfaces the non-ink/proximal side of the display sheet 70 and the second bandage sheet 76 interfaces the opposing electric ink/distal side of the display sheet 70, wherein first bandage sheet 74 engages the area around a predetermined wound site. In use, such an embodiment acts as a sufficient barrier to prevent false alarms based on body moisture, while still enabling positive alarms when a sufficient amount of blood is being discharged from the predetermined wound site. Note body moisture tends to be less mobile through the bandage sheets 74, 76, compared to blood. As a result, a sufficient amount of blood will be able to migrate from the wound site, into the adjacent first bandage sheet 74 (here, again, the first bandage sheet 74 contacts near the patient's wound site, while the non-ink side of the display sheet 70 interfaces the first bandage sheet 74, wherein the ink side of the display sheet 70 is facing away from the patient's skin and interfaces the second bandage sheet 76) and run around to the second bandage sheet 76 so as to operatively engage with the interfacing ink side of the display sheet 70. To facilitate the operability of such an arrangement, the first and second bandage sheets may interface beyond a periphery of the display sheet 70 (but within the periphery defined by the adhesive 78.

The dimensions and shapes of the display sheet 70 and sandwiching bandage sheets 74, 76 may be any size adapted to accommodate various wound sites and locations. Even though only shown in FIG. 4 as rectangular, their shapes and dimension include, but are not limited to, circular, crescent, half circular, and square shapes and the like. In certain embodiments, the dimensions of the rectangular shape may be approximately 1 to 1.5 inches wide by 4-6 inches in length.

The simple combination of the transparent display sheet 70 with electric ink 72 forming the open circuit 40, sandwiched between two bandage sheets 74, 76, facilitates a very inexpensive, adaptable system that can save lives.

It should be understood, of course, that the foregoing relates to exemplary embodiments of the invention and that modifications may be made without departing from the spirit and scope of the invention as set forth in the following claims.

What is claimed is:

1. A device, comprising:
a display sheet having a proximal side and an opposing distal side;
a first and second conductive element disposed along the distal side of the display sheet;
a plurality of first electric ink markings extending from the first conductive element; and
a plurality of second electric ink markings extending from the second conductive element,
wherein none of the plurality of first electric ink markings touch any of the plurality of second electric ink markings.

2. The device of claim 1, wherein the display sheet is transparent and made from flexible material adapted to conform to a body part of a patient.

3. The device of claim 1, wherein each first electric ink marking is spaced apart from each adjacent second electric ink marking.

4. The device of claim 1, further comprising:
a first bandage sheet interfacing the distal side of the display sheet; and
a second bandage sheet interfacing the proximal side of the display sheet.

5. The device of claim 4, wherein the first and second bandage sheets sandwich the display sheet.

6. The device of claim 1, wherein the plurality of first and second electric ink markings and the first and second conductive elements provide an open circuit.

7. The device of claim 6, wherein the open circuit is electrically connected in series with a power supply and a power alarm.

8. A device, comprising:
a transparent display sheet having a proximal side and an opposing distal side, wherein the transparent display sheet is made from a flexible material adapted to conform to a body part of a patient;
a first and second conductive element disposed along the distal side of the display sheet;
a plurality of first electric ink markings extending from the first conductive element;
a plurality of second electric ink markings extending from the second conductive element, wherein the plurality of first and second electric ink markings and the first and second conductive elements provide an open circuit; and
a first bandage sheet and a second bandage sheet sandwiching the display sheet.

9. The method of detecting blood comprising:
providing a transparent display sheet having a proximal side and an opposing distal side, wherein the transparent display sheet is made from a flexible material adapted to conform to a body part of a patient, and wherein a first and second conductive element is disposed along said distal side;
marking said distal side with electric ink so as to configure a desired open circuit, wherein a first portion of the electric ink electrically connects to the first conductive element and a second portion of the electric ink electrically connects to the second conductive element;
electrically connecting the open circuit in series with a power supply and a power alarm; and
placing said proximal side near a wound site.

10. The method of claim 9, further sandwiching the display sheet between a first and second bandage sheet prior to placing said proximal side near the wound site.

11. The method of claim 9, further determining the desired open circuit configuration based in part on the wound site.
